(19) United States
(12) Patent Application Publication
(10) Pub. No.: US 2015/0320570 A1
(43) Pub. Date: Nov. 12, 2015

(54) SPINAL IMPLANTS AND METHODS OF USE THEREOF

(71) Applicant: GLOBUS MEDICAL, INC., Audubon, PA (US)

(72) Inventors: Sean Suh, Jamesburg, NJ (US); Jeff Bennett, Pottstown, PA (US)

(21) Appl. No.: 14/804,485

(22) Filed: Jul. 21, 2015

Related U.S. Application Data
(63) Continuation of application No. 13/150,599, filed on Jun. 1, 2011.
(60) Provisional application No. 61/350,294, filed on Jun. 1, 2010.

(51) Int. Cl. A61F 2/44; A61B 17/70; A61B 17/88; A61F 2/46

(52) U.S. Cl. CPC A61F 2/4405; A61F 2/441; A61F 2/4611; A61B 17/7067; A61B 17/8802; A61F 2002/4475; A61F 2002/4631; A61F 2002/4635

(57) ABSTRACT

Devices and methods for treating one or more damaged, diseased, or traumatized intervertebral discs to reduce or eliminate associated back pain. Specifically, devices and methods of repairing a portion of the spine encompass interspinous spacers, for example, inflated between and secured to one or both of the adjacent spinous processes.

### CROSS REFERENCE TO RELATED APPLICATIONS

This application is a continuation of U.S. patent application Ser. No. 13/150,599, filed Jun. 1, 2011, which claims priority to U.S. provisional application No. 61/350,294, the contents of which are hereby incorporated by reference in their entireties for all purposes.

### FIELD OF THE INVENTION

The invention encompasses devices and methods for treating one or more damaged, diseased, or traumatized portions of the spine, including intervertebral discs, to reduce or eliminate associated back pain. Specifically, the invention encompasses interspinous spacers, intervertebral spacers and corpectomy spacers.

### BACKGROUND OF THE INVENTION

The vertebral column serves as the main structural support of the human skeleton. The vertebral column consists of a number of vertebrae separated by intervertebral discs. A vertebra approximates a cylindrical shape, with wing-like projections and a bony arch. The arches create a passageway through which the spinal cord runs. The vertebral column is held upright by fibrous bands of muscle and ligament. There are seven vertebrae in the cervical region, twelve in the thoracic region, five in the lumbar region, and five in the sacral region that are usually fused together. The integrity of the vertebral column is critical to protecting the fragile spinal cord, in addition to its duties in supporting the skeleton.

When a vertebra becomes damaged or diseased, surgery may be used to replace the vertebra or a portion thereof with a prosthetic device for maintaining the normal spacing of the vertebrae and to support the spine. A prosthesis, which may be referred to as a corpectomy spacer or spinous spacer or implant, can be inserted into the cavity created where the vertebra was removed.

A corpectomy spacer or spinous spacer or implant should be easily adjustable to allow the surgeon to quickly select the height of the device during surgery to fit the needs of the patient. The desired height of the device will depend on the amount of bone that is removed from the patient, the size of the patient, as well as the location of the removed bone (i.e., cervical region or lumbar region). In addition, a one-size-fits-all device may reduce manufacturing costs because fewer different parts and/or models will be required to meet the needs of the marketplace.

While prosthetic corpectomy implants are known in the art, the inventors have developed improved corpectomy implants that are more easily adjusted to achieve the necessary height to replace the excised vertebra during the implantation process, while also possessing the biomechanical properties necessary for long-term implantation in the human body and the immediate fixation ability to provide stability to the spinal column.

### SUMMARY OF THE INVENTION

The inventors have surprisingly found that the interspinous spacer compositions and methods of the invention may overcome the shortcomings associated with currently used replacement and repair technology. As used herein, the terms “interspinous spacer,” “corpectomy spacer,” and “implant” are used interchangeably and refer to the composition of the invention.

Accordingly, in one embodiment, the invention encompasses an expandable spacer comprising: (i) an outer jacket, (ii) one or more central regions located within the outer jacket capable of receiving one or more filler materials, and (iii) a unidirectional valve to allow filling the one or more central regions with the one or more filler materials. In certain exemplary embodiments, the expandable spacer composition is in the form of a balloon, and the filler material fills a central cavity of the expandable spacer composition. In other exemplary embodiments, the balloon is fillable in situ to conform to the dimensions of an intevertebral space of the subject (i.e., the patient).

In another embodiment, the invention encompasses an expandable spacer comprising (i) an outer jacket, (ii) one or more central regions capable of receiving one or more filler materials, (iii) a unidirectional valve to allow filling the central region with the one or more filler materials, and (iv) anchoring elements to secure the spacer to one or more vertebrae. In certain exemplary embodiments, the one or more vertebrae are adjacent to the spacer composition.

In another embodiment, the invention encompasses an expandable spacer comprising (i) an outer jacket, (ii) one or more central regions capable of receiving one or more filler materials, (iii) a unidirectional valve to allow filling with the one or more filler materials, and (iv) one or more bumpers to support compression loading.

In another embodiment, the invention encompasses an expandable spacer comprising:

a. an outer jacket comprised of a biocompatible material;

b. an inner surface capable of being filled with a load bearing polymeric or elastomeric material,

c. a unidirectional valve to allow filling of the inner surface; and

d. a sealing crimp to prevent leakage of the load bearing polymeric or elastomeric material filling the inner surface.

wherein a top surface and/or a bottom surface of the outer jacket are textured to provide anchorage with one or more vertebral endplates. In certain embodiments, the expandable spacer further includes one or more internal or external bumpers to support compression loading.

In another embodiment, the invention encompasses a method of replacing or repairing a vertebral disc comprising:

a. removing a vertebral disc to create a cavity;

b. inserting a expandable spacer composition comprising a finable inner surface into the cavity;

c. filling the inner surface with a load bearing polymeric or elastomeric material; and

d. sealing the expandable spacer to prevent leakage of the load bearing polymeric or elastomeric material filling the inner surface.

### BRIEF DESCRIPTION OF THE FIGURES

A more complete understanding of the present invention may be obtained by reference to the accompanying drawings, when considered in conjunction with the subsequent detailed description. The embodiments illustrated in the drawings are intended only to exemplify the invention and should not be construed as limiting the invention to the illustrated embodiments, in which:

FIG. 1 illustrates a non-limiting, exemplary embodiment of the insertion of a deflated single- or multi-lumen expandable intervertebral, intravertebral, or corpectomy spacer into a vertebral space or cavity using a catheter or endoscope. FIG. 1 further illustrates the inflating of the expandable spacer using mechanical or hydraulic means with an elastomeric or polymeric filler material.

FIG. 2 illustrates a non-limiting, exemplary embodiment of the insertion of a deflated single- or multi-lumen expandable intervertebral, intravertebral, or corpectomy spacer 210 into the vertebral cavity using a catheter or endoscope 220. FIG. 2A illustrates a rolled-up expandable spacer 210 located inside a catheter or endoscope 220. FIG. 2B illustrates an expandable spacer 210. FIG. 2C illustrates the unrolling of the expandable spacer 210. FIG. 2D illustrates an expanded spacer 210, which remains attached to the catheter or endoscope 220 to allow filling with a filler material.

FIG. 3 illustrates a non-limiting, exemplary embodiment of the insertion of another deflated single- or multi-lumen expandable intervertebral, interspinous, or corpectomy spacer 310 before insertion into the interspinous space using a catheter or endoscope 320. FIG. 3 also illustrates the expanded spacer 330 located inside the interspinous space.

FIG. 4a illustrates a non-limiting, exemplary embodiment of the insertion of a deflated single- or multi-lumen expandable intervertebral, interspinous, or corpectomy spacer 410 including insertion holes 420 to allow a screw 430 or bone nail to secure the corpectomy spacer to the vertebra. FIG. 4b illustrates a non-limiting, exemplary embodiment of an expandable spacer 410 secured between two spinous processes of adjacent vertebrae.

FIG. 5 illustrates a non-limiting, exemplary embodiment of the filling of the expandable intervertebral, interspinous, or corpectomy spacer 510 being filled with bone cement 530 or another filler material using an endoscope or catheter 520.

FIG. 6a illustrates a top view a non-limiting, exemplary embodiment of the single- or multi-lumen expandable intervertebral or corpectomy spacer 610 located in the vertebral cavity including one or more outer bumpers 620 and one or more inner bumpers 630 to support compression loading. FIG. 6b illustrates a non-limiting, exemplary embodiment of the single- or multi-lumen expandable spacer 610 located in the vertebral cavity between two vertebrae 600. FIG. 6c illustrates a non-limiting, exemplary expandable spacer 610 comprising one or more outer bumpers 620 and one or more inner bumpers 630 to support compression loading.

FIG. 7a illustrates a non-limiting, exemplary view of an expandable intervertebral or corpectomy spacer 710 located in a vertebral space with a collapse prevention bumper 730. FIG. 7b illustrates a non-limiting, exemplary view of an expandable spacer 710 including one or more keels 720 to anchor to bone and facilitate fixation and one or more inner bumpers 730 to support compression loading, wherein the inner bumper is located within the skin, shell, or jacket of the spacer. FIG. 7c illustrates a non-limiting, exemplary view of an expandable spacer 710 including one or more keels 720 to facilitate fixation and one or more inner bumpers 730 to support compression loading, wherein the inner bumper is located outside the skin or jacket of the spacer. FIG. 7d illustrates a non-limiting, exemplary expandable spacer 710 comprising one or more keels 720 to facilitate fixation and one or more inner bumpers 730 to support compression loading, a unidirectional valve 740 to allow filling with the one or more filler materials and a seal plug 750 to prevent leakage of the filler material.

### DETAILED DESCRIPTION OF THE INVENTION

The invention generally encompasses vertebrae replacement and repair technology.

In one embodiment, the invention encompasses an expandable corpectomy spacer (also referred to herein as an artificial disc or spinous spacer or implant) comprising (i) an outer jacket, (ii) one or more central regions located within the outer jacket capable of receiving one or more filler materials, and (iii) a unidirectional valve to allow filling the one or more central regions with the one or more filler materials.

In certain illustrative embodiments the expandable corpectomy spacer outer jacket is comprised of one or more elastomeric or polymeric materials, a biodegradable or bioresorbable material, or a combination thereof

In certain illustrative embodiments, the polymeric material is polypropylene, polyethylene, polyurethane, polycarbonate urethane, polyetheretherketone (PEEK), polyester, polyethylene terephthalate (PET), poly olefin copolymer, polypropylene, polyethylene or a combination thereof

In certain illustrative embodiments, the biodegradable or bioresorbable material is collagen, cellulose, polysaccharide, polylactic acid (PLA), polyglycolic acid (PGA), polylactic acid/polyglycolic acid, a polylevolactic acid, a polydioxanone (PDA), poly-DL-lactic acid (PDLLA) or a combination thereof

In certain illustrative embodiments, the one or more or elastomeric materials comprise thermoplastic polyurethane elastomer, polysiloxane modified styrene-ethylene/butylene block copolymer, polycarbonate-urethane, polycarbonate-urethane cross-linked by a polyol, silicone rubber, silicone elastomer, polyether urethane, polyester urethane, a polyether polyester copolymer, polypropylene oxide, styrene isoprene butadiene, or combinations thereof.

In certain illustrative embodiments, the expandable corpectomy spacer composition is in the form of a balloon.

In certain illustrative embodiments, the central fillable cavity is pre-shaped with dimensions that conform to an intevertebral disc space.

In certain illustrative embodiments, the central fillable cavity comprises a single lumen.

In certain illustrative embodiments, the central fillable cavity comprises more than one lumen.

In certain illustrative embodiments, the central cavity can be filled with bone cement, a biocompatible fluid or gel, a load-bearing polymeric or elastomeric material, or a combination thereof

In certain illustrative embodiments, the bone cement is polymethylmethacrylate (PMMA).

In certain illustrative embodiments, the biocompatible fluid or gel is saline, beta-glucan, hyaluronic acid and derivatives thereof, polyvinyl pyrrolidone or a hydrogel derivative thereof, polyvinyl acetate, dextrans or a hydrogel derivative thereof, glycerol, polyethylene glycol, block copolymers based on ethylene oxide and propylene oxide), succinaylated collagen, liquid collagen, and other polysaccharides or biocompatible polymers or combinations thereof

In certain illustrative embodiments, the load bearing polymeric or elastomeric material is thermoplastic polyurethane elastomer, polysiloxane modified styrene-ethylene/butylene block copolymer, polycarbonate-urethane, polycarbonate-urethane cross-linked by a polyol, silicone rubber, silicone elastomer, polyether urethane, polyester urethane, a polyether polyester copolymer, polypropylene oxide, silicone, urethane, silicone-urethane copolymer, polycarbonate-urethane copolymer, polyethylene terephthalate, saline, beta-glucan, hyaluronic acid and derivatives thereof, polyvinyl pyrrolidone or a hydrogel derivative thereof, dextrans or a hydrogel derivative thereof, glycerol, polyethylene glycol, succinaylated collagen, liquid collagen, and other polysaccharides or biocompatible polymers or combinations thereof

In certain illustrative embodiments, the outer jacket is porous.

In certain illustrative embodiments, the porous outer jacket comprises one or more bioactive agents, which diffuse into the surrounding tissue after implantation.

In certain illustrative embodiments, the one or more bioactive agents promote growth or reduce inflammation.

In certain illustrative embodiments, the spacer further comprises anchoring elements.

In certain illustrative embodiments, the anchoring elements comprise holes to allow one or more bone screws or nails to secure the spacer to one or more vertebrae.

In another embodiment, the invention encompasses an expandable corpectomy spacer comprising (i) an outer jacket, (ii) one or more central regions capable of receiving one or more filler materials, (iii) a unidirectional valve to allow filling the central region with the one or more filler materials, and (iv) anchoring elements to secure the spacer to one or more vertebrae.

In certain illustrative embodiments, the outer jacket is comprised of one or more elastomeric or polymeric materials, a biodegradable or bioresorbable material, or a combination thereof

In certain illustrative embodiments, the polymeric material is polypropylene, polyethylene, polyurethane, polycarbonate urethane, Polyetheretherketone (PEEK), polyester, PET, poly olefin copolymer, polypropylene, polyethylene or a combination thereof

In certain illustrative embodiments, the biodegradable or bioresorbable material is collagen, cellulose, polysaccharide, polylactic acid (PLA), polyglycolic acid (PGA), polylactic acid/polyglycolic acid, a polylevolactic acid (PPLA), a polydioxanone (PDA), poly-DL-lactic acid (PDLLA) or a combination thereof

In certain illustrative embodiments, the one or more elastomeric materials comprise thermoplastic polyurethane elastomer, polysiloxane modified styrene-ethylene/butylene block copolymer, polycarbonate-urethane, polycarbonate-urethane cross-linked by a polyol, silicone rubber, silicone elastomer, polyether urethane, polyester urethane, a polyether polyester copolymer, polypropylene oxide, styrene isoprene butadiene, or combinations thereof.

In certain illustrative embodiments, the spacer composition is in the form of a balloon.

In certain illustrative embodiments, the central fillable cavity is pre-shaped with dimensions that conform to an intevertebral disc space.

In certain illustrative embodiments, the central fillable cavity comprises a single lumen.

In certain illustrative embodiments, the central fillable cavity comprises a more than one lumen.

In certain illustrative embodiments, the central cavity can be filled with bone cement, a biocompatible fluid or gel or a combination thereof.

In certain illustrative embodiments, the bone cement is polymethylmethacrylate (PMMA).

In certain illustrative embodiments, the biocompatible fluid or gel is saline, beta-glucan, hyaluronic acid and derivatives thereof, polyvinyl pyrrolidone or a hydrogel derivative thereof, polyvinyl acetate, dextrans or a hydrogel derivative thereof, glycerol, polyethylene glycol, block copolymers based on ethylene oxide and propylene oxide), succinaylated collagen, liquid collagen, and other polysaccharides or biocompatible polymers or combinations thereof

In certain illustrative embodiments, the outer jacket is porous.

In certain illustrative embodiments, the porous outer jacket further comprises one or more bioactive agents, which diffuse into the surrounding tissue after implantation.

In certain illustrative embodiments, the one or more bioactive agents promote growth or reduce inflammation.

In another embodiment, the invention encompasses an expandable corpectomy spacer comprising (i) an outer jacket, (ii) one or more central regions capable of receiving one or more filler materials, (iii) a unidirectional valve to allow filling with the one or more filler materials, and (iv) one or more bumpers to support compression loading.

In certain illustrative embodiments, the outer jacket is comprised of one or more elastomeric or polymeric materials, a biodegradable or bioresorbable material, or a combination thereof

In certain illustrative embodiments, the polymeric material is polypropylene, polyethylene, polyurethane, polycarbonate urethane, polyetheretherketone (PEEK), polyester, PET, poly olefin copolymer, polypropylene, polyethylene or a combination thereof

In certain illustrative embodiments, the biodegradable or bioresorbable material is collagen, cellulose, polysaccharide, polylactic acid (PLA), polyglycolic acid (PGA), polylactic acid/polyglycolic acid, a polylevolactic acid (PPLA), a polydioxanone (PDA), poly-DL-lactic acid (PDLLA) or a combination thereof

In certain illustrative embodiments, the one or more or elastomeric materials comprise thermoplastic polyurethane elastomer, polysiloxane modified styrene-ethylene/butylene block copolymer, polycarbonate-urethane, polycarbonate-urethane cross-linked by a polyol, silicone rubber, silicone elastomer, polyether urethane, polyester urethane, a polyether polyester copolymer, polypropylene oxide, styrene isoprene butadiene, or combinations thereof.

In certain illustrative embodiments, the spacer composition is in the form of a balloon.

In certain illustrative embodiments, the central fillable cavity is pre-shaped with dimensions that conform to an intevertebral disc space.

In certain illustrative embodiments, the central fillable cavity comprises a single lumen.

In certain illustrative embodiments, the central fillable cavity comprises a more than one lumen.

In certain illustrative embodiments, the central cavity can be filled with bone cement, a biocompatible fluid or gel or a combination thereof.

In certain illustrative embodiments, the bone cement is polymethylmethacrylate (PMMA).

In certain illustrative embodiments, the biocompatible fluid or gel is saline, beta-glucan, hyaluronic acid and derivatives thereof, polyvinyl pyrrolidone or a hydrogel derivative thereof, polyvinyl acetate, dextrans or a hydrogel derivative thereof, glycerol, polyethylene glycol, block copolymers based on ethylene oxide and propylene oxide), succinaylated collagen, liquid collagen, and other polysaccharides or biocompatible polymers or combinations thereof

In certain illustrative embodiments, the outer jacket is porous.

In certain illustrative embodiments, the porous outer jacket further comprises one or more bioactive agents, which diffuse into the surrounding tissue after implantation.

In certain illustrative embodiments, the one or more bioactive agents promote growth or reduce inflammation.

In certain illustrative embodiments, the spacer further comprises anchoring elements to secure the spacer to one or more vertebrae.

In certain illustrative embodiments, the anchoring elements comprise holes to allow a screw or nail to secure the spacer to one or more vertebrae.

In certain illustrative embodiments, the bumper is in the internal part of the jacket.

In certain illustrative embodiments, the bumper is located on the external part of the jacket.

In another embodiment, the invention encompasses a method of repairing a vertebra comprising:

(i) removing all or a portion of a vertebral disc to create a vertebral cavity;

(ii) inserting an expandable corpectomy spacer comprising one or more fillable central cavities into the vertebral cavity;

(iii) filling the expandable corpectomy spacer with one or more filler materials; and

(iv) sealing the expandable corpectomy spacer to prevent removal of the one or more filler materials.

In certain illustrative embodiments, the removing of the vertebral disc is done using forceps.

In certain illustrative embodiments, the inserting the expandable corpectomy spacer replacement composition is done using an endoscope or catheter.

In certain illustrative embodiments, the expandable corpectomy spacer composition is comprised of one or more biocompatible elastomers comprised of thermoplastic polyurethane elastomer, polysiloxane modified styrene-ethylene/butylene block copolymer, polycarbonate-urethane, polycarbonate-urethane cross-linked by a polyol, silicone rubber, silicone elastomer, polyether urethane, polyester urethane, a polyether polyester copolymer, polypropylene oxide, and combinations thereof

In certain illustrative embodiments, the expandable corpectomy spacer composition is in the form of an inflatable balloon.

In certain illustrative embodiments, the one or more filler materials comprise polymethylmethacrylate, silicone, urethane, silicone-urethane copolymer, polycarbonate-urethane copolymer, polyethylene terephthalate, beta-glucan, hyaluronic acid and derivatives thereof, polyvinyl pyrrolidone or a hydrogel derivative thereof, dextrans or a hydrogel derivative thereof, glycerol, polyethylene glycol, succinaylated collagen, liquid collagen, and other polysaccharides or biocompatible polymers or combinations thereof

In certain illustrative embodiments, the sealing of the expandable corpectomy spacer comprises sutures, adhesives, in-situ fabricated plugs, pre-fabricated plugs, textiles, expandable plugs, or combinations thereof.

### Corpectomy Jacket, Artificial Disc, and Interspinous Spacer Technology of the Invention

The invention generally encompasses expandable spinal implant compositions, including disc replacement compositions that can be implanted with minimally invasive surgical procedures. Due to the composition, make-up and mechanical properties (e.g., flexibility and compressibility), the replacement compositions of the invention will result in less blood loss during implantation, shorter post-operative recovery times, and shorter surgical operation time.

In one embodiment, the invention encompasses a vertebral disc replacement composition including a solid, deformable, load-bearing material capable of being filled with one or more elastomeric or polymeric materials, a biodegradable or bioresorbable material, or a combination thereof

The composition may be useful for treating or replacing one or more herniated or degenerated discs. In an illustrative embodiment, the composition is used in minimally invasive endoscopic discectomy (e.g., lumbar discectomy) for treating or replacing one or more herniated or degenerated discs. The disc replacement composition can maintain its structural and functional integrity. To repair an injury, the disc material is removed in a minimally invasive surgical operation to form a cavity. This may be carried out with, for example, a forceps-like instrument.

In certain illustrative embodiments, the implant incorporates a deflated deformable, load-bearing material (e.g., a single or multi-lumen elastomeric balloon), which can be inflated with one or more elastomeric or polymeric materials, a biodegradable or bioresorbable material, or a combination thereof

In certain illustrative embodiments, the disc replacement composition can mimic a disc of a healthy subject and will bear physiologic loads through stiffness imparted by the one or more elastomeric or polymeric materials, a biodegradable or bioresorbable material, or a combination thereof. The stiffness and internal hydrostatic pressure can assist load bearing, support the spine from all sides and prevent creep or effusion and stress relaxation of the elastomeric material.

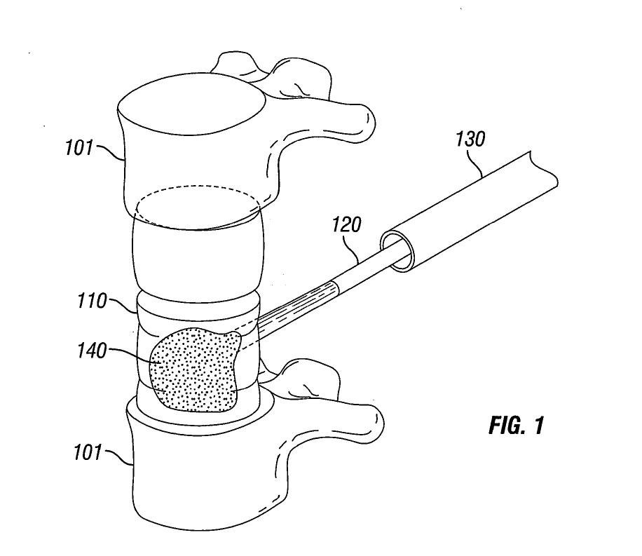

FIG. 1 illustrates a non-limiting, exemplary embodiment of the insertion of an intervertebral, intravertebral, or corpectomy spacer 110 using a cannulated tube 120 and a delivery tube 130 and inserting into an intervertebral space between two vertebrae 101 and filling the spacer with a filler material 140. In FIG. 1, a deflated single- or multi-lumen corpectomy spacer 110 can be inserted into the intervertebral cavity 105 using a catheter or endoscope 120. FIG. 1 illustrates the inflation or filling of the spacer using mechanical or hydraulic means with load bearing filler material 140.

FIG. 1 also illustrates the inflated disc replacement composition arranged between two vertebrae. It is understood that the upper vertebra rests with its lower end plate in a surface-to-surface manner in the same way as the lower vertebra with its upper end plate against the intervertebral disc.

The disc replacement composition comprising a solid, deformable, load-bearing material can be comprised of any durable material that is safe for in vivo transplantation including, but not limited to, one or more biocompatible polymers of elastomers including thermoplastic polyurethane elastomer, polysiloxane modified styrene-ethylene/butylene block copolymer, polycarbonate-urethane, polycarbonate-urethane cross-linked by a polyol, silicone rubber, silicone elastomer, polyether urethane, polyester urethane, a polyether polyester copolymer, polypropylene oxide, and combinations thereof

In certain illustrative embodiments, any material that is safe for in vivo use can be used including, but not limited to, silicone, urethane, silicone-urethane copolymer, polycarbonate-urethane copolymer, polyethylene terephthalate, or combinations thereof

In other illustrative embodiments, the filler material that is injected in the composition includes, but is not limited to, saline, beta-glucan, hyaluronic acid and derivatives thereof, polyvinyl pyrrolidone or a hydrogel derivative thereof, dextrans or a hydrogel derivative thereof, glycerol, polyethylene glycol, succinaylated collagen, liquid collagen, and other polysaccharides or biocompatible polymers, alcohols, polyols, amino acids, sugars, proteins, polysaccharides, chondroitin sulfate, dermatan sulfate, heparin sulfate, biglycan, syndecan, keratocan, decorin, aggrecan, and combinations thereof

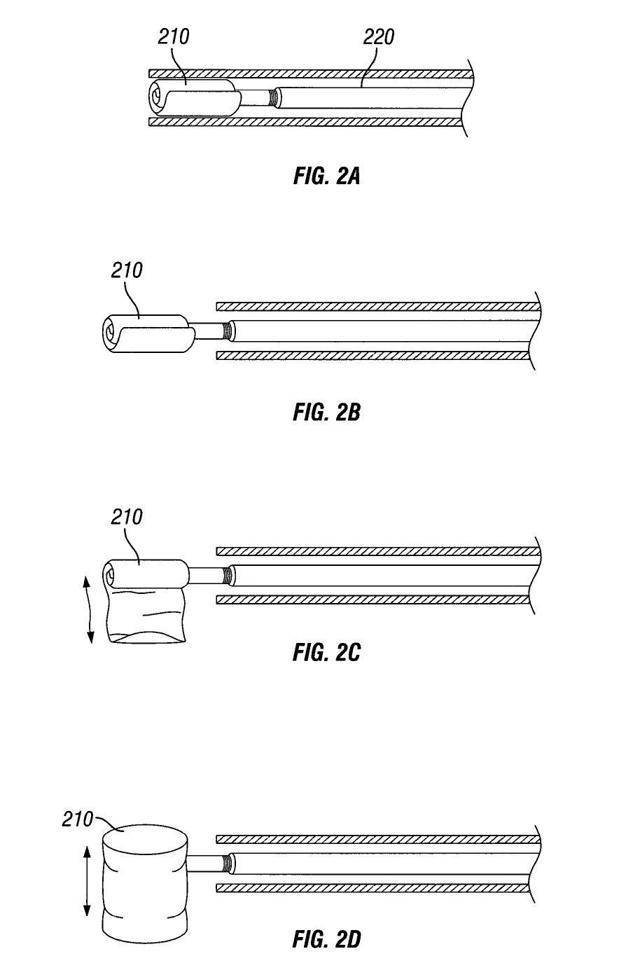

FIG. 2 illustrates a representation of the steps of inserting the spacer of FIG. 1 followed by filling the spacer. In a first step, a spacer is delivered to an intervertebral space using a catheter or endoscope and a delivery tube. In an illustrative embodiment, the spacer is initially deflated and for example rolled to allow easy insertion. The spacer is then deployed to the intervertebral space and then filled to provide support.

FIG. 2 illustrates a non-limiting, exemplary blown up view of the insertion of a deflated single- or multi-lumen balloon 210 into the cavity using a catheter or endoscope 220. FIG. 2 further illustrates the inflating of the spacer using mechanical or hydraulic means with load bearing material.

Additionally, the spacer or jacket surface can be coated with one or more bioactive agents. “Bioactive agents,” as used herein, include, but are not limited to, chemotactic agents; therapeutic agents (e.g., antibiotics, steroidal and non-steroidal analgesics and anti-inflammatories (including certain amino acids such as glycine), anti-rejection agents such as immunosuppressants and anti-cancer drugs); various proteins (e.g., short term peptides, bone morphogenic proteins, collagen, hyaluronic acid, glycoproteins, and lipoprotein); cell attachment mediators; biologically active ligands; integrin binding sequence; ligands; various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin derived growth factor (e.g., IGF-1, IGF-II) and transforming growth factors (e.g., TGF-.beta.I-III), parathyroid hormone, parathyroid hormone related peptide, bone morphogenic proteins (e.g., BMP-2, BMP-4; BMP-6; BMP-7; BMP-12; BMP-13; BMP-14), sonic hedgehog, growth differentiation factors (e.g., GDF5, GDF6, GDF8), recombinant human growth factors (e.g., MP52, and MP-52 variant rhGDF-5), cartilage-derived morphogenic proteins (CDMP-1; CDMP-2, CDMP-3)); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments and DNA plasmids; and combinations thereof. Suitable effectors likewise include the agonists and antagonists of the agents described above. The growth factor can also include combinations of the growth factors described above. In addition, the growth factor can be autologous growth factor that is supplied by platelets in the blood. In this case, the growth factor from platelets will be an undefined cocktail of various growth factors. If other such substances have therapeutic value in the orthopedic field, it is anticipated that at least some of these substances will have use in the present invention, and such substances should be included in the meaning of “bioactive agent” and “bioactive agents” unless expressly limited otherwise. Illustrative examples of preferred bioactive agents include culture media, bone morphogenic proteins, growth factors, growth differentiation factors, recombinant human growth factors, cartilage-derived morphogenic proteins, hydrogels, polymers, antibiotics, anti-inflammatory medications, immunosuppressive mediations, autologous, allogenic or xenologous cells such as stem cells, chondrocytes, fibroblast and proteins such as collagen and hyaluronic acid. Bioactive agents can be synthetic (e.g., bioactive glass), autologus, allogenic, xenogenic or recombinant.

In another embodiment, the invention encompasses an implant that can replace a herniated or degenerated disc. In certain embodiments, the herniated or degenerated disc is in the early stages of degenerative disc disease. In various embodiments, the implant is composed of a polymeric or elastomeric material that has the mechanical properties that mimic the vertebral disc of a healthy subject.

Accordingly, the implant can be composed of a material including, but not limited to, one or more biocompatible polymers of elastomers including thermoplastic polyurethane elastomer, polysiloxane modified styrene-ethylene/butylene block copolymer, polycarbonate-urethane, polycarbonate-urethane cross-linked by a polyol, silicone rubber, silicone elastomer, polyether urethane, polyester urethane, a polyether polyester copolymer, polypropylene oxide, and combinations thereof

In certain embodiments, the implant is composed of a polymeric or elastomeric material that is compressible and flexible to allow insertion and implantation endoscopically without causing the implant to substantially lose shape or form.

In other embodiments, the implant is composed of a polymeric or elastomeric material that is porous. Bioactive agents as defined herein can be loaded into the implant, for example, to promote growth or to alleviate pain associated with degeneration.

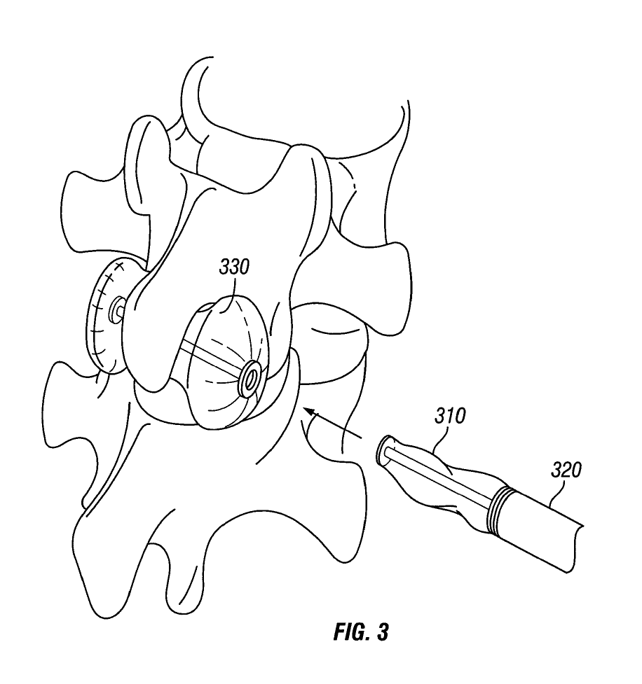

FIG. 3 illustrates a non-limiting example of another embodiment of an expandable spacer for use in the interspinous space. In certain embodiments, a deflated spacer 310 may be attached to an endoscope or catheter 320 and once inflated, spacer 330 present in the interspinous space. In certain embodiments, the composition has the same height as the intended disc height to be restored. One skilled in the art will also consider the cross section of the replacement composition since contact surface area help with load/force distribution in the spine.

In other illustrative embodiments, to achieve a desired disc height the more than one spring nucleus replacement composition can be inserted into the vertebral cavity, for example, in layers. In certain embodiments, the implant composition comprises a single biocompatible polymeric or elastomeric material that is solid, deformable, and load-bearing and comprises a center cavity and one or more envelope cavities surrounding the center cavity. In certain embodiments, the center cavity and one or more envelope cavities surrounding the center cavity can be independently filled with a plurality of elastomeric or polymeric materials.

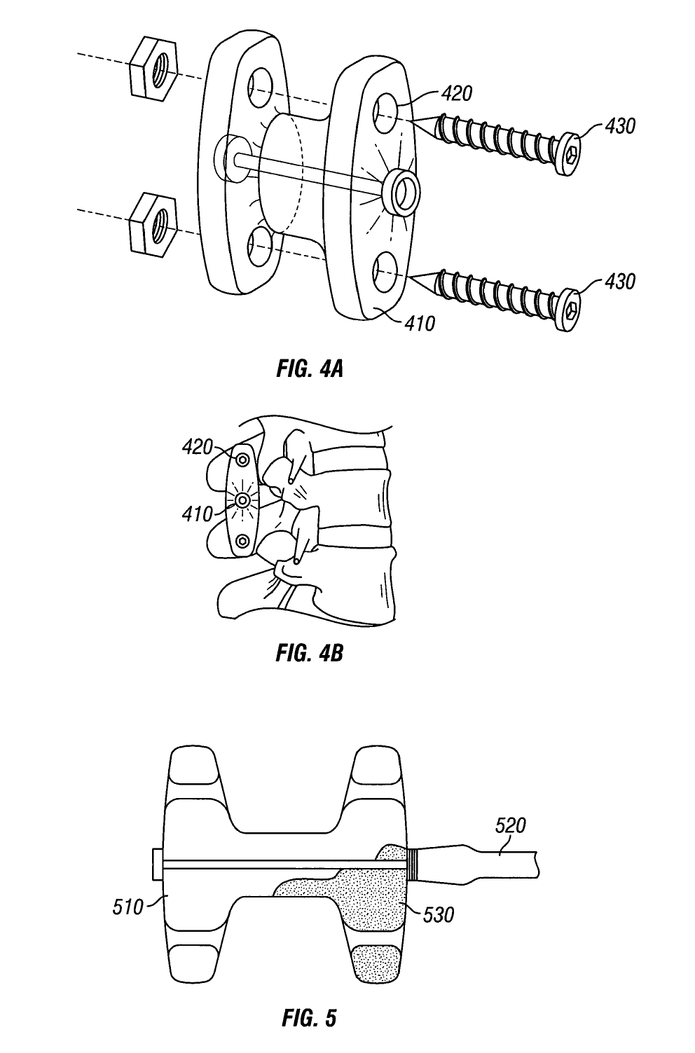

FIG. 4a illustrates a non-limiting, exemplary interspinous spacer replacement composition 410 including holes 420 that allow a bone screw or nail 430 to secure the replacement composition to vertebrae. FIG. 4b illustrates a side perspective view of the implant composition secured to a spinous process.

FIG. 5 illustrates a non-limiting, exemplary inflated interspinous spacer 510 attached to an endoscope or catheter 520. FIG. 5 illustrates the spacer being filled with a filler material. The filler material may be chosen from known materials to achieve the desirous mechanical properties of the spacer. For a more rigid implant, for example, a cement product may be inserted into spacer 510. For a more compliant implant, a gel or the like may be used.

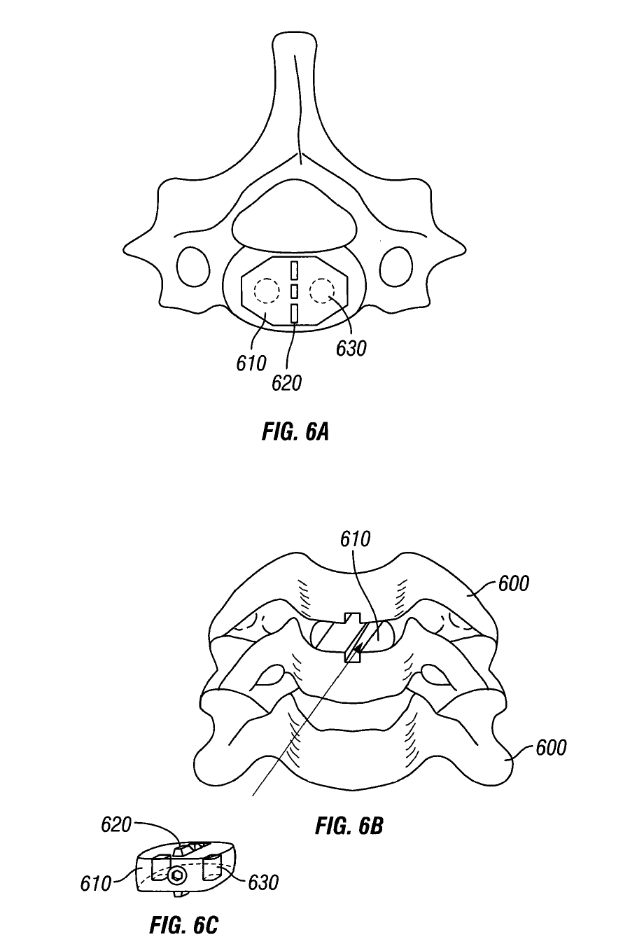

FIG. 6 illustrates a non-limiting, exemplary expandable corpectomy spacer 610 comprising one or more outer bumpers 620 and one or more inner bumpers 630 to support compression loading.

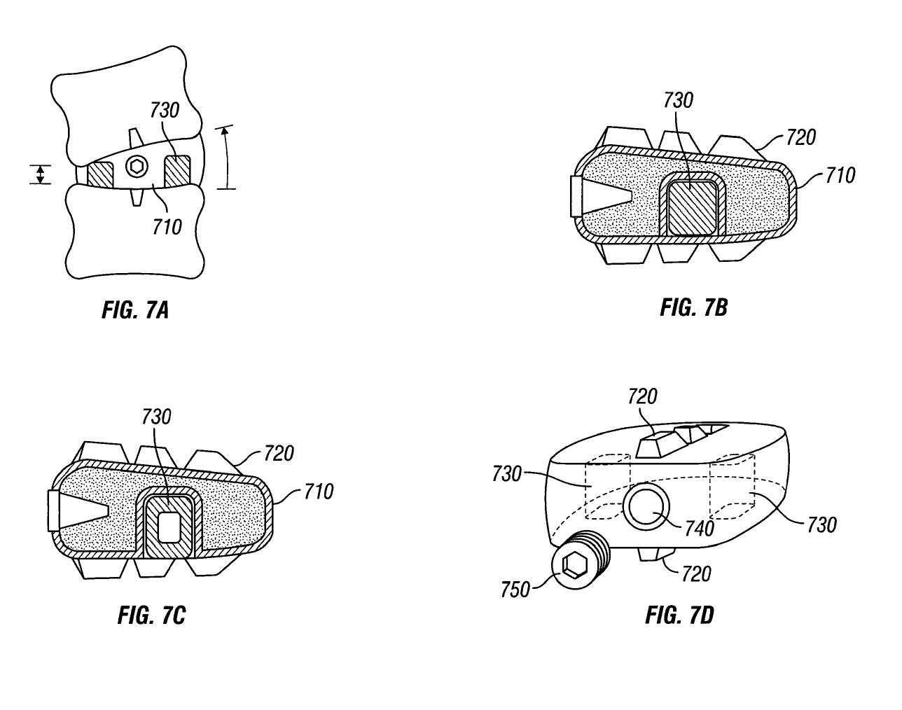

FIG. 7A illustrates a non-limiting, exemplary expandable intervertebral or corpectomy or intervertebral spacer including a bumper located inside the jacket, shell or outer perimeter thereof. FIG. 7b illustrates a non-limiting, exemplary expandable spacer including a bumper located inside the jacket of the spacer. FIG. 7c illustrates a non-limiting, exemplary expandable spacer including one or more keels located outside the jacket of the spacer. In certain embodiments, the composition has the same height as the intended disc height to be restored. One skilled in the art will also consider the cross section of the disc replacement composition since contact surface area helps with load/force distribution in the spine. FIG. 7d illustrates a non-limiting, exemplary expandable spacer 710 comprising one or more keels 720 to facilitate fixation and one or more inner bumpers 730 to support compression loading, a unidirectional valve 740 to allow filling with the one or more filler materials and a seal plug 750 to prevent leakage of the filler material.

In certain embodiments, the expandable implant includes a textured top and/or bottom surface to provide anchorage with vertebral endplates and an optionally textured surface along the curved perimeter. The implant can be filled with a load bearing polymeric or elastomeric material to allow the implant to conform to the shape of the vertebral cavity. In an illustrative, non-limiting embodiment, the implant is further comprised of a unidirectional valve for filling the inner surface; and a sealing crimp to prevent leakage of the load bearing polymeric or elastomeric material filling the inner surface.

One illustrative embodiment encompasses a corpectomy spacer comprising:

a. an outer jacket comprised of a biocompatible material;

b. an inner surface capable of being filled with a load bearing polymeric or elastomeric material,

c. a unidirectional valve for filling the inner surface; and

d. a sealing crimp to prevent leakage of the load bearing polymeric or elastomeric material filling the inner surface.

wherein the outer jacket has a cylindrical-like shape, wherein a top surface and/or a bottom surface are textured to provide anchorage with vertebral endplates.

Another illustrative embodiment encompasses a disc replacement composition comprising:

a. an inflatable outer jacket comprised of a biocompatible material;

b. one or more inner surfaces located in the outer jacket capable of being filled with a load bearing polymeric or elastomeric material,

c. a unidirectional valve for filling the inner surface; and

d. a sealing crimp to prevent leakage of the load bearing polymeric or elastomeric material filling the inner surface.

wherein the outer shell has a cylindrical-like shape, wherein a top surface and/or a bottom surface include bumpers so that compression loading is supported thereby reducing the risk of burst due to uncontrolled pressure.

In certain illustrative embodiments, the outer jacket is comprised of (1) metals (e.g., titanium or titanium alloys, alloys with cobalt and chromium, cobalt-chrome, stainless steel); (2) plastics (e.g., ultra-high molecular weight polyethylene (UHMWPE), polymethylmethacrylate (PMMA), polytetrafluoroethylene (PTFE), polyetheretherketone (PEEK), nylon, polypropylene, and/or PMMA/polyhydroxy-ethylmethacrylate (PHEMA)); (3) ceramics (e.g., alumina, beryllia, calcium phosphate, and/or zirconia, among others); (4) composites; and/or the like. In certain embodiments, the materials may be partially or completely bio-resorbable as desired or appropriate.

In other illustrative embodiments, the containment shell can include a partially or totally textured surface to allow anchorage with the vertebral endplates. As used herein, textured, refers to any grooved or rough texture (e.g., a Velcro®-like texture) or porous features that increases the friction and anchorage with the vertebral endplates.

Another embodiment encompasses a disc replacement system including an outer jacket having a cylindrical shape and a textured top and bottom surface to provide anchorage with vertebral endplates. The implant can be filled with a load bearing polymeric or elastomeric material filling to allow the implant to conform to the shape of the disc cavity. The illustrative, non-limiting implant includes a polymer jacket (e.g., urethanes, silicones), or a combination thereof, a unidirectional valve for filling the inner surface; and a sealing crimp to prevent leakage of the load bearing polymeric or elastomeric material filling the inner surface.

In certain embodiments, the load bearing polymeric or elastomeric material is a thermoplastic polyurethane elastomer, polysiloxane modified styrene-ethylene/butylene block copolymer, polycarbonate-urethane, polycarbonate-urethane cross-linked by a polyol, silicone rubber, silicone elastomer, polyether urethane, polyester urethane, a polyether polyester copolymer, polypropylene oxide, silicone, urethane, silicone-urethane copolymer, polycarbonate-urethane copolymer, polyethylene terephthalate, saline, beta-glucan, hyaluronic acid and derivatives thereof, polyvinyl pyrrolidone or a hydrogel derivative thereof, dextrans or a hydrogel derivative thereof, glycerol, polyethylene glycol, succinaylated collagen, liquid collagen, and other polysaccharides or biocompatible polymers or combinations thereof

Generally, the jacket includes a unidirectional valve to allow filling of the containment shell with the load bearing polymeric or elastomeric material. In addition, the nucleus containment shell includes a sealing crimp to prevent leakage of the load bearing polymeric or elastomeric material.

In another embodiment, the invention encompasses a disc repair system comprising:

a disc replacement composition comprising:

an outer surface comprised of a biocompatible material and adapted to conform to an inner wall of a vertebral cavity and comprising a valve attached to the outer surface comprising a rigid socket geometry; and

an inner surface having a central recess capable of receiving a load bearing polymeric or elastomeric material,

wherein the outer and inner surfaces define a solid, deformable thickness therebetween.

In certain embodiments, the repair system includes a guide for inserting the disc replacement composition.

The disc replacement composition can be comprised of any durable material that is safe for in vivo transplantation including, but not limited to, one or more biocompatible polymers of elastomers including thermoplastic polyurethane elastomer, polysiloxane modified styrene-ethylene/butylene block copolymer, polycarbonate-urethane, polycarbonate-urethane cross-linked by a polyol, silicone rubber, silicone elastomer, polyether urethane, polyester urethane, a polyether polyester copolymer, polypropylene oxide, and combinations thereof

In certain illustrative embodiments, the elastomer includes any material that is safe for in vivo use including, but not limited to, silicone, urethane, silicone-urethane copolymer, polycarbonate-urethane copolymer, polyethylene terephthalate, or combinations thereof.

In other illustrative embodiments, the biocompatible filler includes any material that is safe for in vivo use including, but not limited to, saline, beta-glucan, hyaluronic acid and derivatives thereof, polyvinyl pyrrolidone or a hydrogel derivative thereof, dextrans or a hydrogel derivative thereof, glycerol, polyethylene glycol, Pluronic® type block copolymers (i.e., based on ethylene oxide and propylene oxide), succinaylated collagen, liquid collagen, and other polysaccharides or biocompatible polymers or combinations thereof. In other embodiments, the biocompatible fluid or gel. includes salts, alcohols, polyols, amino acids, sugars, proteins, polysaccharides, chondroitin sulfate, dermatan sulfate, heparin sulfate, biglycan, syndecan, keratocan, decorin, aggrecan, and combinations thereof. In other embodiments, the filler includes in situ curable materials, for example, polyurethanes and silicones) that will form a solid in situ.

### Kits

The invention also contemplates kits including a disc replacement composition and the equipment and materials required to insert the composition into the intervertebral cavity.

Accordingly, the disc replacement composition can be manufactured in varying widths, lengths, and dimensions to accommodate the type of surgery and needs of the surgeon.

In addition, the kits can also include the load bearing polymeric or elastomeric material including a plurality of elastomeric materials and the necessary cannulas to administer them.

The kits of the invention are intended to broaden a surgeon's options once in surgery to provide a patient with the most optimal nucleus replacement composition and annulus fibrosus repair technology.

### EXAMPLES

### Example 1

To repair a herniated disk injury, vertebral disc material is removed in a surgical operation to form a cavity. This may be carried out with, for example, a forceps-like instrument with which the jelly-like nucleus material is cut off and the opening may also be enlarged and its edges smoothed. The thus removed nucleus material may be used for growing a culture of the patient's own body cells.

A disc replacement composition of the invention is then inserted into the cavity. The disc replacement composition includes, for example, a biocompatible solid, deformable, load-bearing material in the form of a balloon, which is deflated and incorporated into the vertebral cavity using a catheter and is selected in relation to the size of the opening such that upon introducing the disc replacement composition into the opening, the opening is not unnecessarily enlarged. The disc replacement composition is connectable by a rod to a handle which can be removed, for example, by unscrewing.

After insertion of the disc replacement composition, the composition can be filled with, for example, an elastomeric or polymeric material. The amount of material can be determined by the surgeon during surgery and depends on the patient's physiology, the location on the vertebra of the implant, and other mechanical and physical properties apparent to the surgeon.

In this way, the entire material of the plug may be flexible or elastic, but it is also possible for the material of the plug to become progressively firmer. When the opening has been closed in this way, cell material grown outside of the body (e.g., in a culture) can be introduced into the interior of the intervertebral disc. For example, this is carried out approximately weeks after the surgical operation described above. Alternatively, the porous jacket can be coated with a bioactive agent that promotes cell growth or provides a therapeutic effect.

In the specification, there have been disclosed typical illustrative embodiments of the invention and, although specific terms are employed, they are used in a generic and descriptive sense only and not for purposes of limitation. Obviously many modifications and variations of the invention are possible in light of the above teachings. It is therefore to be understood that the invention may be practiced otherwise than as specifically described.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited above are incorporated herein by reference to the extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A method of repairing a portion of a spine comprising:
inserting a deflated interspinous spacer comprising one or more fillable central cavities into an interspinous space between spinous processes of adjacent vertebrae;
filling the one or more fillable central cavities with one or more filler materials to inflate the interspinous spacer;
sealing the interspinous spacer to retain the one or more filler materials within the interspinous spacer; and
attaching the interspinous spacer to one or both of the spinous processes of the adjacent vertebrae.

2. The method of claim 1, wherein the interspinous spacer includes a first end portion, a central portion, and a second end portion, wherein the first and second end portions are enlarged relative to the central portion.

3. The method of claim 1, wherein the interspinous spacer includes one or more holes in the first and second end portions.

4. The method of claim 3, wherein attaching the interspinous spacer to the adjacent vertebrae includes inserting an anchoring element into at least one of the one or more holes and into one of the spinous processes.

5. The method of claim 4, wherein the anchoring element extends through a hole in the first end portion, through one of the spinous processes, and through a hole in the second end portion of the interspinous spacer.

6. The method of claim 4, wherein the anchoring element is a bone screw.

7. The method of claim 1, wherein the interspinous spacer is attached to both of the spinous processes of the adjacent vertebrae.

8. The method of claim 1, further comprising, before inserting the interspinous spacer into the interspinous space, attaching a catheter or endoscope to the deflated interspinous spacer.

9. The method of claim 1, wherein the interspinous spacer is implanted using a minimally invasive surgical (MIS) procedure.

10. The method of claim 1, wherein the interspinous spacer includes a unidirectional valve to allow filling of the one or more central cavities with the one or more filler materials.

11. The method of claim 1, wherein the one or more filler materials include bone cement, a biocompatible fluid or gel, a load-bearing polymeric or elastomeric material, or a combination thereof.

12. A method of implanting an interspinous spacer comprising:
attaching a catheter or endoscope to a deflated interspinous spacer;
inserting the deflated interspinous spacer comprising one or more fillable central cavities into an interspinous space between spinous processes of adjacent vertebrae;
filling the one or more fillable central cavities with one or more filler materials to inflate the interspinous spacer;
attaching the interspinous spacer to one or both of the spinous processes of the adjacent vertebrae; and
removing the catheter or endoscope from the interspinous spacer.

13. The method of claim 12, wherein the interspinous spacer has a first end, a central portion, and a second end portion, wherein the first and second end portions are enlarged relative to the central portion.

14. The method of claim 13, wherein attaching the interspinous spacer to the adjacent vertebrae includes attaching a first anchoring element between the first and second end portions of the interspinous spacer to one of the spinous processes and attaching a second anchoring element between the first and second end portions of the interspinous spacer to the other of the spinous processes.

15. The method of claim 12, wherein the interspinous spacer is implanted using a minimally invasive surgical (MIS) procedure.

16. The method of claim 12, wherein the one or more filler materials include bone cement, a biocompatible fluid or gel, a load-bearing polymeric or elastomeric material, or a combination thereof.

17. A method of securing an interspinous spacer to a spine comprising:
inserting an interspinous spacer into an interspinous space between spinous processes of adjacent vertebrae;
inflating the interspinous spacer having a first end portion defining a first hole and a second hole, a central portion, and a second end portion defining a first hole and a second hole, wherein the first and second end portions are enlarged relative to the central portion; and
securing the interspinous spacer to one of the spinous processes by inserting a first anchoring element through the first hole in the first end portion of the interspinous spacer, through the spinous process, and through the first hole in the second end portion of the interspinous spacer.

18. The method of claim 17, further comprising securing the interspinous spacer to the other spinous process by inserting a second anchoring element through the second hole in the first end portion of the interspinous spacer, through the other spinous process, and through the second hole in the second end portion of the interspinous spacer.

19. The method of claim 17, further comprising, before inserting the interspinous spacer into the interspinous space, attaching a catheter or endoscope to the interspinous spacer.

20. The method of claim 17, wherein the interspinous spacer is implanted in a deflated state using a minimally invasive surgical (MIS) procedure.
